(19) United States
(12) Patent Application Publication
(10) Pub. No.: US 2023/0382827 A1
(43) Pub. Date: Nov. 30, 2023

(54) Production of Alkylaromatic Compounds

(71) Applicant: ExxonMobil Chemical Patents Inc., Baytown, TX (US)

(72) Inventors: Aaron W. Peters, New Hope, PA (US); Reuben J. Britto, Seattle, WA (US); William J. Knaeble, Golden Valley, MN (US); Ivy D. Johnson, Lawrenceville, NJ (US); Christopher G. Oliveri, Erie, CO (US)

(21) Appl. No.: 18/248,965

(22) PCT Filed: Sep. 30, 2021
(86) PCT No.: PCT/US2021/052895

§ 371 Date: Apr. 13, 2023

Related U.S. Application Data
(60) Provisional application No. 63/110,606, filed on Nov. 6, 2020.

(51) Int. Cl. C07C 6/12; B01J 29/08; B01J 37/00; B01J 35/10

(52) U.S. Cl. CPC C07C 6/126; B01J 29/08; B01J 37/0018; B01J 35/1019; B01J 35/1023; B01J 35/1042; B01J 35/1038; C07C 2529/08

(57) ABSTRACT

A process for producing a mono-alkylated benzene comprises contacting benzene with di-alkylated benzene(s) and/or tri-alkylated benzene(s) in the presence of a transalkylation catalyst composition under transalkylation conditions to convert at least part of the di-alkylated benzene(s) and tri-alkylated benzene(s) to mono-alkylated benzene. The transalkylation catalyst composition comprises a treated zeolitic material having increased mesoporous surface area compared to the precursor catalyst composition from which it is made.

### CROSS-REFERENCE TO RELATED APPLICATION

This application claims priority to and the benefit of U.S. Provisional Application No. 63/110,606 having a filing date of Nov. 6, 2020, the disclosure of which is incorporated herein by reference in its entirety.

### FIELD

This disclosure relates to a process for producing alkylaromatic compounds, particularly ethylbenzene and cumene.

### BACKGROUND

Ethylbenzene and cumene are valuable commodity chemicals which are used industrially for the production of styrene monomer and the coproduction of phenol and acetone, respectively. Ethylbenzene and cumene are typically produced by alkylating benzene with a C₂ or C₃ alkylating agent, such as ethylene or propylene, under liquid phase or mixed gas-liquid phase conditions in the presence of an acid catalyst, particularly a zeolite catalyst. In addition to the desired monoalkylated product, the process inevitably produces the dialkylated and trialkylated analogs as well as other heavy by-products. Thus, to maximize the yield of ethylbenzene and cumene, it is conventional to transalkylate the polyalkylated products with benzene to generate additional monoalkylated product. The product of the transalkylation reaction is then fed, together with the alkylation reaction effluent, to one or more benzene columns, to recover unreacted benzene, then to one or more EB or cumene columns, to recover the desired monoalkylated product.

Current state-of-the-art transalkylation catalyst compositions employ as the active materials metallosilicate zeolites having channels and/or surface pockets channel defined by 12-membered rings of tetrahedrally coordinated atoms (see, for example, International Patent Publication No. WO2018/140149). These zeolites can convert polyalkylbenzene molecules to ethylbenzene and cumene with high selectivity and activity. However, these catalysts require elevated temperatures, low flow rates of the substrate feed, and large catalyst beds to achieve sufficient conversion, all of which serve to increase process costs. Moreover, the problem is exacerbated by the fact that the trialkylated species are significantly less reactive than the dialkylated species so that, with existing catalysts, it is difficult to find a processing window where effective conversion of the trialkylated species is achieved without adversely affecting the selectivity of the conversion of the dialkylated species.

There is therefore significant interest in providing transalkylation catalyst compositions which can operate at lower temperatures without sacrificing conversion activity and mono-alkylated benzene selectivity.

### SUMMARY

It has now been found that certain zeolitic materials that have been treated to generate mesoporosity in addition to their inherent long range crystallinity are effective at relatively low temperatures in catalyzing the transalkylation of benzene with di-alkylated benzene(s) and/or tri-alkylated benzene(s), particularly di-isopropylbenzene(s) and/or tri-isopropylbenzene(s), at high conversion rates and with high selectivity to the desired monoalkylated species, particularly cumene.

Accordingly, a first aspect of this disclosure relates to a transalkylation process. The process can comprise one or more of the following steps: (I) providing a precursor catalyst composition exhibiting a first mesoporous surface area of al m²/g; (II) treating the precursor catalyst composition to obtain a treated precursor catalyst composition, wherein the treated precursor catalyst composition exhibits a second mesoporous surface area of a2 m²/g, and wherein a2>a1, preferably 10%≤(a2−a1)/a1*100%≤1000%; (III) forming a transalkylation catalyst composition from the treated precursor catalyst composition; (IV) feeding a transalkylation feed mixture comprising (i) benzene and (ii) di-alkylated benzene(s) and/or tri-alkylated benzene(s) into a transalkylation zone; and (V) contacting the transalkylation feed mixture with the transalkylation catalyst composition in the transalkylation zone under transalkylation conditions to produce a transalkylation effluent rich in mono-alkylated benzene(s) relative to the transalkylation feed mixture.

A second aspect of this disclosure relates to a process for producing a mono-alkylated benzene. The process can comprise one or more of the following steps: (a) contacting a feedstream comprising benzene with an alkylating agent in the presence of an alkylation catalyst composition under alkylation conditions effective to convert at least part of the benzene in the feedstream to the desired mono-alkylated benzene and produce an alkylation effluent comprising the mono-alkylated benzene, di-alkylated benzene(s) and tri-alkylated benzene(s); (b) separating the alkylation effluent into a first fraction containing the mono-alkylated benzene and a second fraction containing di-alkylated benzene(s) and tri-alkylated benzene(s); (c) contacting at least part of the second fraction with benzene in the presence of a transalkylation catalyst composition under transalkylation conditions including a temperature from 100 to 300° C. effective to convert at least part of the di-alkylated benzene(s) and tri-alkylated benzene(s) to the mono-alkylated benzene and produce a transalkylation effluent, wherein the transalkylation catalyst composition is obtained by: (c1) providing a precursor catalyst composition exhibiting a first mesoporous surface area of al m²/g; (c2) treating the precursor catalyst composition to obtain a treated precursor catalyst composition, wherein the treated precursor catalyst composition exhibits a second mesoporous surface area of a2 m²/g, and wherein a2≤a1, preferably 10%≤(a2−a1)/a1*100%≤1000%; and (c3) forming a transalkylation catalyst composition from the treated precursor catalyst composition;

### BRIEF DESCRIPTION OF THE DRAWINGS

FIG. 1 is a graph of differential pore volume against pore diameter comparing the pore size distribution of the comparative transalkylation catalyst composition of comparative Example 5 (comprising a low activity faujasite, a precursor catalyst composition) with the pore size distribution of the inventive transalkylation catalyst compositions of Examples 6 to 8 (each comprising a treated precursor catalyst composition made from the precursor catalyst composition of comparative Example 5 to increase its mesoporosity).

FIG. 2 shows the X-ray diffraction (“XRD”) patterns in which normalized intensity is plotted against 2-theta (2θ) of the catalyst compositions of comparative Example 1 (comprising a high activity faujasite, a precursor catalyst composition), Example 4 (comprising a treated precursor catalyst composition made from the precursor catalyst composition of comparative Example 1 to increase its mesoporosity), comparative Example 5 (comprising a low activity faujasite, a precursor catalyst composition), and Example 8 (comprising a treated precursor catalyst composition made from the precursor catalyst composition of comparative Example 5 to increase its mesoporosity).

FIG. 3 is a graph in which the relative selectivity towards cumene is plotted against the temperature needed to achieve 50% conversion of di-isopropylbenzene (DIPB) for the catalyst compositions of Examples 1 to 9 when used in the transalkylation tests described in Example 10.

FIG. 4 is a graph in which the relative conversion of tri-isopropylbenzene (TIPB) at 50% conversion of DIPB is plotted against the temperature needed to achieve 50% DIPB conversion for the catalyst compositions of Examples 1 to 9 when used in the transalkylation tests described in Example 10.

### DETAILED DESCRIPTION

Unless otherwise indicated, the terms “mesoporous” and “mesoporosity” are used in the art-recognized sense as referring to a porous material comprising pores with an intermediate size, that is with at least one cross-sectional dimension ranging anywhere from about 20 to about 500 Å.

The terms “zeolite” and “zeolitic material” are used herein in the manner defined by the International Zeolite Association Constitution (Section 1.3) to include both natural and synthetic zeolites as well as molecular sieves and other porous materials having related properties and/or structures. The term “zeolite” also refers to a group, or any member of a group, of structured aluminosilicate minerals comprising cations such as sodium and calcium or, less commonly, barium, beryllium, lithium, potassium, magnesium and strontium; characterized by the ratio (Al+Si):O=approximately 1:2, an open tetrahedral framework structure capable of ion exchange, and loosely held water molecules that allow reversible dehydration. The term “zeolite” also includes “zeolite-related materials” or “zeotypes” which are prepared by replacing Si⁴⁺or Al³⁺with other elements as in the case of aluminophosphates (e.g., MeAPO, SAPO, ElAPO, MeAPSO, and ElAPSO), gallophosphates, zincophophates, and titanosilicates.

Zeolites exhibit long-range crystallinity, by which is meant they comprise one or more phases having repeating structures, referred to as unit cells, that repeat in a space for at least 10 nm. In one embodiment, the zeolites after modification and extrusion of this invention still exhibit long range crystallinity and order (i.e., the have peaks in the XRD that can be ascribed to the parent zeolite). The zeolitic materials employed in producing the transalkylation catalyst compositions disclosed herein exhibit microporosity, that is contain pores with at least one cross-sectional dimension less than 20 Å, such as less than 10 Å. In addition, the initial zeolitic materials employed in producing the transalkylation catalyst compositions disclosed herein may, but are not required to, exhibit some mesoporosity. In some embodiments, the initial zeolitic material may have a ratio of mesoporous surface area to microporous surface area less than 0.8, such as less than 0.7, such as less than 0.6, prior to any treatment to enhance the mesoporosity of the material. Preferably, the initial zeolitic material has a framework structure selected from the group consisting of FAU, BEA, MOR, MWW and mixtures thereof, with FAU being particularly preferred.

### I. The First Aspect of this Disclosure

Described herein is a transalkylation process and/or a process for producing mono-alkylated benzenes, such as ethylbenzene and cumene, in which benzene is contacted with a mixture comprising di-alkylated benzene(s) and/or tri-alkylated benzene(s) in the presence of a transalkylation catalyst composition under transalkylation conditions effective to convert at least part of the dialkylated benzene(s) and tri-alkylated benzene(s) to mono-alkylated benzene, wherein the transalkylation catalyst composition comprises a treated precursor catalyst composition (e.g., a treated zeolitic material) having mesoporosity (preferably in addition to long range crystallinity) and wherein the treated precursor catalyst composition (e.g., a treated zeolitic material) exhibits a higher mesoporous surface area than the precursor catalyst composition prior to the treatment. The treatment method can comprise treating the precursor catalyst composition having long range crystallinity with at least a surfactant under conditions effective to generate or increase mesoporosity in the precursor catalyst composition. A non-limiting example of the precursor catalyst is an initial zeolitic material.

The precursor catalyst composition can comprise, consist essentially of, or consist of a catalytically active component such as an initial zeolitic material. Additionally or alternatively, the precursor catalyst composition can comprise a first auxiliary component, such as a co-catalyst, a second catalytically active component, or a catalytically inert component.

Non-limiting examples of the first auxiliary component are additional molecular sieves which may, but are not required to, be capable of catalyzing a reaction in the transalkylation zone. Such molecular sieves can comprise one or more zeolites. Non-limiting examples of useful molecular sieves for the first auxiliary component in the precursor catalyst composition can include large pore molecular sieves having a Constraint Index less than 2, and mixtures and combinations thereof. Suitable large pore molecular sieves include zeolite beta, zeolite Y, Ultrastable Y (USY), Ultrahydrophobic Y (UHP-Y), Dealuminized Y (Deal Y), mordenite, ZSM-3, ZSM-4, ZSM-14, ZSM-18, ZSM-20 and mixtures thereof. Zeolite Beta is described in U.S. Pat. No. 3,308,069, and Re. No. 28,341. Zeolite ZSM-3 is described in U.S. Pat. No. 3,415,736. Zeolite ZSM-4 is described in U.S. Pat. No. 4,021,947. Zeolite ZSM-14 is described in U.S. Pat. No. 3,923,636. Zeolite ZSM-18 is described in U.S. Pat. No. 3,950,496. Zeolite ZSM-20 is described in U.S. Pat. No. 3,972,983. Low sodium Ultrastable Y molecular sieve (USY) is described in U.S. Pat. Nos. 3,293,192 and 3,449,070. Ultrahydrophobic Y (UHP-Y) is described in U.S. Pat. No. 4,401,556. Dealuminized Y zeolite (Deal Y) may be prepared by the method found in U.S. Pat. No. 3,442,795. Zeolite Y and mordenite are naturally occurring materials but are also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104.

Another class of molecular sieve materials which may be present in the precursor catalyst composition as a first auxiliary component is the group of inherently mesoporous crystalline materials exemplified by the MCM-41 and MCM-48 materials. These mesoporous crystalline materials are described in U.S. Pat. Nos. 5,098,684; 5,102,643; and 5,198,203. MCM-41, which is described in U.S. Pat. No. 5,098,684, is characterized by a microstructure with a uniform, hexagonal arrangement of pores with diameters of at least about 1.3 nm: after calcination it exhibits an X-ray diffraction pattern with at least one d-spacing greater than about 1.8 nm and a hexagonal electron diffraction pattern that can be indexed with a d100 value greater than about 1.8 nm which corresponds to the d-spacing of the peak in the X-ray diffraction pattern. The preferred catalytic form of this material is the aluminosilicate although other metallosilicates may also be utilized. MCM-48 has a cubic structure and may be made by a similar preparative procedure.

Still another non-limiting example of the first auxiliary component in the precursor catalyst composition is a binder or a matrix material. Non-limiting examples of the binder include silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof. Specific examples of the binder include, but are not limited to, kaolin, bentonite, and mixtures and combinations thereof. The binder may be a naturally occurring (with or without enhancing treatment) or a synthetic material. The binder can function to increase the mechanical property of the precursor catalyst composition.

The precursor catalyst composition can consist essentially of or consist of the treated precursor catalyst and the optional additional molecular sieves, substantially free or totally free of a binder as described above.

The precursor catalyst composition may have been formed into any desired geometry and/or size, in such non-limiting forms as powder, pellets, extrudates, and the like.

The precursor catalyst composition can, but are not required to, have one or more of the following features: (i) a silica (SiO₂) to alumina (Al₂O₃) molar ratio of r3 to r4, where r3 and r4 can be, independently, e.g., 10, 15, 20, 25, 30, 35, 40, 45, 50, 55, 60, 65, 70, 75, 80, 90, 95, 100, as long as r3<r4; (ii) a total surface area of s(t)3 to s(t)4 m²/g, where s(t)3 and s(t)4 can be, independently, e.g., 200, 250, 300, 350, 400, 450, 500, 550, 600, 650, 700, 750, 800, 850, 900, 950, 1,000, as long as s(t)3<s(t)4; and (iii) a micropore surface area of s(mp)3 to s(mp)4 m²/g, where s(mp)3 and s(mp)4 can be, independently, e.g., 50, 100, 150, 200, 250, 300, 350, 400, 450, 500, 550, 600, 650, 700. In certain embodiments, including but not limited to those where the precursor catalyst composition comprises a zeolitic material such as a faujasite, the precursor catalyst composition may have a mesopore surface area) of from s(e)3 to s(e)4 m²/g, where s(e)3 and s(e)4 can be, independently, e.g., 10, 15, 20, 25, 30, 35, 40, 45, 55, 60, 65, 70, 75, 80, 85, 90, 95, 100, 110, 120, 130, 140, 150, 160, 180, 200, 250, 300, 350, 400, as long as s(e)3<s(e)4.

One method for treating the precursor catalyst composition can comprise the following steps: (a) treating the precursor catalyst composition with a surfactant; (b) treating the precursor catalyst composition with an acid before or after step (a); and (c) treating the precursor catalyst composition with a base after step (a) and/or step (b) above.

US2013/0183231 Al discloses treatment processes for introducing mesopores into a zeolitic material to enlarge its mesoporous surface area using a combination of acid treatment, surfactant treatment, followed by an alkaline solution treatment, the content of which is incorporated herein by reference in its entirety. The various processes disclosed in US2013/0183231 A1 may be used to obtain the transalkylation catalyst composition from a precursor catalyst composition comprising a zeolite. Furthermore, U.S. Patent Application Publication No. 2007/0244347, the contents of which is incorporated herein by reference, discloses processes for generating or enlarging mesoporisity in a zeolitic material, which may be used for making the transalkylation catalyst composition in the process of this disclosure.

In certain specific embodiments, in preparing the transalkylation catalyst composition for use in the present process, the initial zeolitic material can first optionally be combined with water to form an initial slurry. In one or more embodiments, the initial zeolitic material can be present in the optional initial slurry in an amount in the range of from about 1 to about 50 weight percent, such as from about 5 to about 40 weight percent, for example from about 10 to about 30 weight percent, such as from about 15 to about 25 weight percent.

In certain specific embodiments, the initial zeolitic material (optionally as part of an initial slurry) is then contacted with a surfactant, typically a cationic surfactant. In one or more embodiments, the surfactant employed can comprise one or more alkyltrimethyl ammonium salts and/or one or more dialkyldimethyl ammonium salts. Suitable salts include cetyltrimethyl ammonium bromide, cetyltrimethyl ammonium chloride and mixtures thereof. In other embodiments, the surfactant comprises a non-ionic surfactant. Examples of suitable commercially available non-ionic surfactants include, but are not limited to, Pluronic™ surfactants (e.g., Pluronic P123™), available from BASF.

In certain specific embodiments, prior to or after addition of the surfactant, an acid can be combined with the initial zeolite material to form, after addition of the surfactant, a treatment mixture comprising the acid, the surfactant, and the zeolitic material. Acids suitable for use herein can be any organic or inorganic (mineral) acids. In various embodiments, the acid employed in this step of the treatment process can be a dealuminating acid. In further embodiments, the acid can also be a chelating agent. Specific examples of acids suitable for use include, but are not limited to, hydrochloric acid, sulfuric acid, nitric acid, acetic acid, sulfonic acid, oxalic acid, citric acid, ethylenediaminetetraacetic acid, tartaric acid, malic acid, glutaric acid, succinic acid, and mixtures of two or more thereof. In certain embodiments, the treatment mixture is prepared in the absence or substantial absence of hydrofluoric acid. As used herein, the term “substantial absence” means a concentration of less than 10 parts per million by weight (“ppmw”).

In various embodiments, the amount of acid employed in the treatment mixture can be in the range of from about 1 to about 10, such as from about 2 to about 8, or about 3 to about 6, milliequivalents per gram of the above-described initial zeolitic material. Typically, the amount of acid employed is such that the treatment mixture has a pH in the range of from about 2 to about 6, or in the range of from about 3 to about 4.

In certain specific embodiments, the resulting treatment mixture can then be agitated for a period of time ranging from about 1 minute to about 24 hours, such as from about 5 minutes to about 12 hours, for example from about 10 minutes to about 6 hours, or from about 30 minutes to about 2 hours. Furthermore, the treatment mixture can be heated (in the presence or absence of agitation) to a temperature from about 30 to about 100° C., such as from about 40 to about 80° C., for a period of time ranging from about 30 minutes to about one week, such as from about an hour to about 2 days.

In certain specific embodiments, following treatment with the above-described acid and surfactant, at least a portion of the resulting surfactant-treated zeolitic material is recovered from the treatment mixture. Any known solid/liquid separation technique, such as filtration, can be used to effect the recovery, thereafter the recovered surfactant-treated zeolitic material can be washed (e.g., with deionized water) one or more times.

In certain specific embodiments, once the surfactant-treated zeolitic material has been recovered from the treatment mixture, it can be contacted with a base. Suitable bases include NaOH, NH₄OH, KOH, Na₂CO₃, TMAOH, and mixtures thereof. In certain embodiments, the base employed can be in the form of an aqueous solution having a concentration in the range of from 0.2 to 15 weight percent. Additionally, the amount of base employed can be such that the base is present at a ratio with the initial quantity of the initial zeolitic material in a range from about 0.1 to about 20 mmol per gram of initial zeolitic material, such as from about 0.1 to about 5 mmol per gram of initial zeolitic material, for example of from about 0.9 to about 4 mmol per gram of initial zeolitic material. Treatment of the surfactant-treated zeolitic material with a base can be performed under elevated temperature conditions, including a temperature from about 30 to about 200° C., such as from about 50 to about 150° C., for a time from about 1 minute to about 2 days, such as from about 30 minutes to about 1 day, for example from about 2 hours to about 20 hours, or from about 16 to about 18 hours.

In certain specific embodiments, following treatment with a base, at least a portion of the resulting mesoporous zeolitic material can be separated from the basic treatment mixture. For example, the mesoporous zeolitic material can be filtered, washed, and/or dried. In one or more embodiments, the mesoporous zeolitic material can be filtered via vacuum filtration and washed with water. Thereafter, the recovered mesoporous zeolitic material can optionally be filtered again and optionally dried.

The product of the treatment method described above is a treated zeolitic material having long range order or crystallinity (that is, exhibit peaks in the X-ray diffraction pattern that can be ascribed to the parent zeolite) and increased mesoporosity as compared with initial zeolitic material. Prior to the treatment step, the precursor catalyst composition exhibits an mesoporous surface area of al m²/g. The treatment results in an increased mesoporous surface area of the treated precursor catalyst composition of a2 m²/g, where a2>a1. While in general a large increase of mesoporous surface area is desirable, in certain embodiments, x1%≤(a2−a1)/a1*100%≤x2%, where x1 and x2, can be, independently, e.g., 10, 20, 30, 40, 50, 60, 70, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as x1<x2. Preferably x1=30 and x2=800. Preferably x1=40 and x2=500. More preferably x1=50 and x2=300. In some embodiments, the ratio of the mesoporous surface area to the microporous surface area of the treated zeolite is at least 10%, such as at least 20%, such as at least 30%, such as at least 40%, such as at least 50%, greater than that of the initial zeolitic material. In certain embodiments, the treated zeolitic material has a ratio of mesoporous surface area to microporous surface area greater than 0.8, such as greater than 0.9, such as greater than 1.

After the treatment of the precursor catalyst composition, a transalkylation catalyst composition can be formed from the treated precursor catalyst composition. In certain embodiments, this forming step can comprise (1) combining the treated precursor catalyst composition with a second auxiliary component; and (2) obtaining the transalkylation catalyst composition from the combined mixture from step (1). The second auxiliary component can include one or more of a co-catalyst, a second catalytically active component different from the catalytic component in the treated precursor catalyst composition, or a catalytically inert component.

Non-limiting examples of the second auxiliary component are additional molecular sieves which may be capable of catalyzing a reaction in the transalkylation zone. The additional molecular sieves can, but are not required to, be treated in a manner similar to the treatment process described above for enlarging mesoporous surface area thereof. Such molecular sieves can comprise one or more zeolites. Non-limiting examples of useful molecular sieves for the second auxiliary component can include large pore molecular sieves having a Constraint Index less than 2, and mixtures and combinations thereof. Suitable large pore molecular sieves include zeolite beta, zeolite Y, Ultrastable Y (USY), Ultrahydrophobic Y (UHP-Y), Dealuminized Y (Deal Y), mordenite, ZSM-3, ZSM-4, ZSM-14, ZSM-18, ZSM-20 and mixtures thereof. Zeolite Beta is described in U.S. Pat. No. 3,308,069, and Re. No. 28,341. Zeolite ZSM-3 is described in U.S. Pat. No. 3,415,736. Zeolite ZSM-4 is described in U.S. Pat. No. 4,021,947. Zeolite ZSM-14 is described in U.S. Pat. No. 3,923,636. Zeolite ZSM-18 is described in U.S. Pat. No. 3,950,496. Zeolite ZSM 20 is described in U.S. Pat. No. 3,972,983. Low sodium Ultrastable Y molecular sieve (USY) is described in U.S. Pat. Nos. 3,293,192 and 3,449,070. Ultrahydrophobic Y (UHP-Y) is described in U.S. Pat. No. 4,401,556. Dealuminized Y zeolite (Deal Y) may be prepared by the method found in U.S. Pat. No. 3,442,795. Zeolite Y and mordenite are naturally occurring materials but are also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104.

Another class of molecular sieve materials which may be used as the second auxiliary component in the present transalkylation catalyst compositions is the group of inherently mesoporous crystalline materials exemplified by the MCM-41 and MCM-48 materials. These mesoporous crystalline materials are described in U.S. Pat. Nos. 5,098,684; and 5,198,203. MCM-41, which is described in U.S. Pat. No. 5,098,684, is characterized by a microstructure with a uniform, hexagonal arrangement of pores with diameters of at least about 1.3 nm: after calcination it exhibits an X-ray diffraction pattern with at least one d-spacing greater than about 1.8 nm and a hexagonal electron diffraction pattern that can be indexed with a d100 value greater than about 1.8 nm which corresponds to the d-spacing of the peak in the X-ray diffraction pattern. The preferred catalytic form of this material is the aluminosilicate although other metallosilicates may also be utilized. MCM-48 has a cubic structure and may be made by a similar preparative procedure. Another non-limiting example of the second auxiliary component in the transalkylation catalyst composition useful for the processes of this disclosure is a binder or a matrix material. Non-limiting examples of the binder include silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof. Specific examples of the binder include, but are not limited to, kaolin, bentonite, and mixtures and combinations thereof. The binder may be a naturally occurring (with or without enhancing treatment) or a synthetic material. The binder can function to increase the mechanical property of the transalkylation catalyst composition. In certain embodiments, the binder can be present at an amount of from c(b)1 to c(b)2 wt %, based on the total weight of the transalkylation catalyst composition, where c(b)1 and c(b)2 can be, independently, e.g., 1, 2, 3, 4, 5, 6, 7, 8, 9, 10, 15, 20, 25, 30, 35, 40, 50, 55, 60, 65, 70, 75, 80, 85, 90, 95, as long as c(b)1<c(b)2.

The transalkylation catalyst composition can consist essentially of or consist of the treated precursor catalyst and the optional additional molecular sieves, substantially free or totally free of a binder as described above. Such binder-free molecular sieve catalyst composition is sometimes called “self-bound catalyst.”

In the forming step, the treated precursor catalyst composition, or the combined mixture of the treated precursor catalyst composition and the auxiliary component, can be formed into any desired geometry and/or size, in such non-limiting forms as powder, pellets, extrudates, and the like. Optionally, a drying and/or calcination step may be carried out to the formed combined mixture to produce the transalkylation catalyst composition.

In certain embodiments, the transalkylation catalyst composition useful in the processes of this disclosure can have one or more of the following features: (i) a total surface area of s(t)1 to s(t)2 m²/g, where s(t)1 and s(t)2 can be, independently, e.g., 200, 250, 300, 350, 400, 450, 500, 550, 600, 650, 700, 750, 800, as long as s(t)1<s(t)2; (ii) a micropore surface area of s(mp)1 to s(mp)2 m²/g, where s(mp)1 and s(mp)2 can be, independently, e.g., 50, 100, 150, 200, 250, 300, 350, 400, 450, 500, 550, 600; and (iii) a mesoporous surface area of s(e)1 to s(e)2 m²/g, where s(e)1 and s(e)2 can be, independently, e.g., 55, 60, 65, 70, 75, 80, 85, 90, 100, 120, 140, 150, 160, 180, 200, 220, 240, 250, 260, 280, 300, 350, 400, 450, 500, 550, 600, 650, as long as s(e)1<s(e)2.

In the transalkylation step, the transalkylation catalyst composition may be present in the transalkylation zone in a fixed bed, a moving bed, a slurry, and the like, suitable for the conversion reactions under the transalkylation conditions. In certain embodiments, the transalkylation conditions can comprise at least one of the following: (i) a temperature in a range from T1 to T2° C., where T1 and T2 can be, independently, e.g., 100, 120, 140, 150, 160, 170, 180, 190, 200, 210, 220, 230, 240, 250, 260, 270, 280, 300, 350, as long as T1<T2; (ii) an absolute pressure in a range from p1 to p2 kilopascal, where p1 and p2 can be, independently, e.g., 100, 200, 300, 400, 500, 600, 700, 800, 900, 1000, 1500, 2000, 3000, 4000, 5000, as long as p1<p2; (iii) a molecular hydrogen (H2) concentration in the hydrocarbon feed in a range from c(H2)1 to c(H2)2 ppm by weight, based on the total weight of the hydrocarbon feed, where c(H2)1 and c(H2)2 can be, independently, e.g., 0, 1, 3, 4, 5, 6, 7, 8, 9, 10, 15, 20, 30, 40, 50, 60, 70, 80, 90, 100, 150, 200, 250, 300, 350, 400, 450, 500, as long as c(H2)1<c(H2)2. Preferably c(H2)2=200. Preferably c(H2)2=100. Preferably c(H2)2=50. Preferably c(H2)2=10. Preferably no H2 is co-fed into the transalkylation zone; and (iv) a WHSV of the hydrocarbon feed in a range from w1to w2 hr⁻¹, where w1 and w2 can be, independently, e.g., 0.5, 1, 1.5, 2, 2.5, 3, 3.5, 4, 5, 6, 7, 8, 9, 10, 11, 12, 13, 14, 15, 16, 17, 18, 19, 20, as long as w1<w2. In the transalkylation zone, the aromatic hydrocarbons may be present in vapor phase and/or liquid phase. Advantageously, =25%, or =30%, or =40%, or =50%, or =60%, or =70%, or =80%, or =90%, or =95%, or =98%, or even substantially all, of the aromatic hydrocarbons are present in liquid phase in the transalkylation zone. In certain specific embodiments, suitable transalkylation conditions for (i) a hydrocarbon feed comprising polyethylbenzenes and benzene and (ii) a hydrocarbon feed comprising poly-isopropylbenzenes with benzene in the presence of a transalkylation catalyst composition described herein include a temperature of 100° C. to 300° C., a pressure of 696 kPa-a to 5100 kPa-a, a weight hourly space velocity of 0.5 to 200 hr⁻¹ based on the weight of polyalkylated aromatic compounds and a benzene/poly-alkylated benzene(s) weight ratio 0.5:1 to 20:1. Preferred conditions include a temperature of 150° C. to 250° C., a pressure of 696 kPa-a to 4137 kPa-a, a weight hourly space velocity of 0.5 to 100 hr⁻¹ based on the weight of polyalkylated aromatic compounds and benzene/poly-alkylated benzene(s) weight ratio 1:1 to 10:1. In one preferred embodiment, where the transalkylation feed comprises di-isopropylbenzene(s) and tri-isopropylbenzene(s) and the desired monoalkylated product comprises cumene, the transalkylation conditions comprise a temperature in the range of 150 to 200° C. Typically, the transalkylation conditions are controlled such that the polyalkylated aromatic compounds and the benzene are at least partially or predominantly in the liquid phase.

A transalkylation catalyst composition fabricated by treating a precursor catalyst composition followed by optional catalyst composition forming as described above can exhibit an increased performance at least in terms of selectivity for the desired compound (e.g., a mono-alkyl aromatic hydrocarbon such as ethylbenzene or cumene) in the transalkylation step compared to a comparative catalyst composition formed from the precursor catalyst composition under the same transalkylation conversion conditions. Thus, in the transalkylating step comprising contacting a hydrocarbon feed with the transalkylation catalyst composition under a set of transalkylation conditions to produce a conversion product, a selectivity for the desired compound (e.g., a mono-alkyl aromatic hydrocarbon such as ethylbenzene or cumene) of sel(pX)2 wt % can be obtained. In contrast, in a reference step (S-ref) below, selectivity for the same desired compound of sel(pX)1 wt %, where sel(pX)1<sel(pX)2, is obtained: (S-ref) contacting the hydrocarbon feed with the comparative catalyst composition in the conversion zone under the same set of transalkylation conditions to produce a reference conversion product. Desirably and advantageously,

$$1 \% \leq \frac{( sel ( pX ) 2 - sel ( pX ) 1}{sel ( pX ) 1} \times 100 \% \leq y 2 \% ,$$

where y1 and y2 can be, independently, e.g., 3, 4, 5, 6, 7, 8, 9, 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as y1<y2. Without intending to be bound by a particular theory, it is believed that the enlarged mesoporous surface area in the transalkylation catalyst composition improves the catalytic activity compared to the comparative catalyst composition.

In certain specific embodiments, depending on the composition of the hydrocarbon feed and the transalkylation conditions employed, the transalkylation catalyst composition employed herein may be effective in converting at least 50% by weight, preferably at least 75% by weight, of di-alkylated benzene(s) in the feed to the equivalent mono-alkylated benzene product and converting at least 25% by weight, preferably at least 50% by weight, of tri-alkylated benzene(s) in the feed at 50 weight % di-alkylated benzene(s) conversion. Typically with the weight ratio of the conversion of tri-alkylated benzene(s) to the conversion of dialkylated benzene(s) is at least 0.2, such as from 0.2 to 2, for example 0.2 to 1.2.

Any mixture of di-alkylated benzene(s) and tri-alkylated benzene(s) can be used in the present transalkylation process, although in most practical embodiments the poly-alkylated benzene(s) feedstock used herein will comprise part or all of the heavy fraction remaining after separation of a desired monoalkylated product, especially ethylbenzene or cumene, from the reaction effluent of the alkylation of benzene with an alkylating agent, especially a C₂ or C₃ alkylating agent. In such a case, the poly-alkylated benzene(s) feedstock will typically contain from 40% by weight to 85% by weight of the di-alkylated benzene(s) and from 5% by weight to 60% by weight, or from 15% by weight to 60% by weight, of the tri-alkylated benzene(s).

### II. The Second Aspect of this Disclosure

Thus, in a second aspect, this disclosure relates to a process for producing a mono-alkylated benzene, in which a feedstream comprising benzene is initially contacted with an alkylating agent in the presence of an alkylation catalyst composition under alkylation conditions effective to convert at least part of the benzene in the feedstream to the desired mono-alkylated benzene and produce an alkylation effluent comprising mono-alkylated benzene, di-alkylated benzene(s) and tri-alkylated benzene(s). The alkylation effluent is then separated into a first fraction containing the mono-alkylated benzene and a second fraction containing the di-alkylated benzene(s) and the tri-alkylated benzene(s). At least part of the second fraction is then contacted with additional benzene in the presence of the transalkylation catalyst composition as described above in connection with the first aspect of this disclosure to convert at least part of the di-alkylated benzene(s) and tri-alkylated benzene(s) to mono-alkylated benzene and produce a transalkylation effluent, from which the mono-alkylated benzene can be recovered.

The above process can find utility with a wide range of alkylating agents, but has particular advantage with C₂ and C₃ alkylating agents. Suitable alkylating agents are olefins and alcohols, which may be linear, branched or cyclic. In some embodiments, the alkylating agent is a C₂ alkylating agent, such as ethylene, or a C₃ alkylating agent, such as propylene and/or isopropanol. Preferably, the alkylating agent comprises propylene and/or isopropanol and the desired mono-alkylated benzene product comprises cumene.

Suitable alkylation catalyst compositions comprises any or all of the molecular sieves discussed above in relation to the transalkylation catalyst, including a zeolitic material which has been treated to enhance its mesoporosity. In addition, the alkylation catalyst may comprise at least one medium pore molecular sieve having a Constraint Index of 2-12 (as defined in U.S. Pat. No. 4,016,218). Suitable medium pore molecular sieves include ZSM-5, ZSM-11, ZSM-12, ZSM-22, ZSM-23, ZSM-35, and ZSM-48. ZSM-5 is described in detail in U.S. Pat. Nos. 3,702,886 and Re. 29,948. ZSM-11 is described in detail in U.S. Pat. No. 3,709,979. ZSM-12 is described in U.S. Pat. No. 3,832,449. ZSM-22 is described in U.S. Pat. No. 4,556,477. ZSM-23 is described in U.S. Pat. No. 4,076,842. ZSM-35 is described in U.S. Pat. No. 4,016,245. ZSM-48 is more particularly described in U.S. Pat. No. 4,234,231.

Other suitable molecular sieves for use as alkylation catalysts in the present process include molecular sieves of the MCM-22 family As used herein, the term “molecular sieve of the MCM-22 family” (or “material of the MCM-22 family” or “MCM-22 family material” or “MCM-22 family zeolite”) includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Molecular sieves of the MCM-22 family include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Materials of the MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No 6,077,498), ITQ-2 (described in International Patent Publication No. WO97/17290), MCM-36 (described in U.S. Pat. No. 5,250,277), MCM-49 (described in U.S. Pat. No. MCM-56 (described in U.S. Pat. No. 5,362,697), UZM-8 (described in U.S. Pat. No. 6,756,030), UZM-8HS (described in U.S. Pat. No. 7,713,513) and mixtures thereof.

Preferred alkylation catalysts comprise zeolite beta or a zeolite of the MCM-22 family The above molecular sieves may be used as the alkylation catalyst without any binder or matrix or can be combined with any of the binder materials discussed above as suitable for use in the transalkylation catalyst.

The reaction conditions used to conduct the alkylation step will depend on the particular alkylating agent employed, but suitable conditions are well within the ambit of anyone of ordinary skill in the art. For example, alkylation of benzene with ethylene to produce ethylbenzene is typically conducted at a temperature about 120° C. to 300° C., preferably, a temperature of from about 150° C. to 260° C., a pressure of 500 to 8300 kPa-a, preferably, a pressure of 1500 to 4500 kPa-a, so that at least part of the reaction mixture is maintained in the liquid phase during the process. Generally, the molar ratio of benzene to ethylene is from about 1 to about 100, preferably from about 20 to about 80. In the case of alkylation of benzene with propylene to produce cumene, typical reaction conditions include a temperature of about 20° C. to about 350° C., for example about 50° C. to about 300° C., such as about 100° C. to 280° C., and a pressure of about 100 kPa to about 20,000 kPa, for example about 500 kPa to about 10,000 kPa, so that at least part of the reaction mixture is maintained in the liquid phase during the process. Generally, the molar ratio of benzene to propylene is maintained within the range of about 1:1 to about 30:1, typically from 1.1:1 to 10:1.

In addition to the desired monoalkylated aromatic product, the effluent from the main alkylation reaction may contain significant quantities of unreacted benzene, together with smaller quantities of polyalkylated species, for example di-isopropylbenzene(s) (DIPB) and some tri-isopropylbenzene (TIPB) in a cumene process, and di-ethylbenzene(s) (DEB) and some tri-ethylbenzene(s) (TEB) in an ethylbenzene process. The effluent from the main alkylation reaction is therefore fed to a separation system to allow recovery of the monoalkylated aromatic product and further processing of the by-products and impurities.

The separation system may include one or more benzene distillation columns, where unreacted benzene may be removed from the effluent as an overhead or side stream for recycle to the alkylation reaction and/or to the transalkylation reactor (as described above). The bottoms from the benzene column(s) can then be fed to one or more monoalkylate distillation columns to recover the desired monoalkylated aromatic product. The bottoms from the monoalkylate column(s) contain the majority of the byproducts of the alkylation reaction heavier than the desired monoalkylate product. This bottoms stream may then be fed to one or more polyalkylate distillation columns to separate a polyalkylated aromatic product stream containing most of the dialkylated by-product and part of the trialkylated by-product for passage to the transalkylation reaction. The remainder of the trialkylated by-product and essentially all of the compounds heavier than the trialkylated by-product may be discharged at the bottoms of the polyalkylate column as residue.

In some embodiments, where the benzene feedstream to the alkylation and/or transalkylation reaction comprises impurities, including, but is not limited to, compounds having at least one of the following elements: nitrogen, halogens, oxygen, sulfur, arsenic, selenium, tellurium, phosphorus, and Group 1 through Group 12 metals. In such a case, the alkylation and/or transalkylation step may further comprise: contacting the benzene feedstream with an absorbent under conditions effective to remove at least part of the impurities. The adsorbent may have catalytic activity and may comprise a molecular sieve, such as any of the molecular sieves described above, and a small quantity of alkylating agent may be simultaneously fed to the adsorbent to react with the benzene feed and thereby act as a marker for poison capacity of the adsorbent.

The invention will now be more particularly described with reference to the following non-limiting Examples and the accompanying drawings.

### Examples 1 to 4

A commercially available high activity faujasite zeolite was used as a base zeolite (a precursor catalyst composition), which was treated to produce three different batches of faujasite crystals (treated precursor catalyst compositions) with varying mesopore surface areas. Four different catalyst compositions were produced by (i) mixing each of the base zeolite and the three treated batches of zeolites with an identical commercially available alumina binder, with a zeolite/binder weight ratio of 65/35; (ii) extruding the mixture into 1/20″ quadralobes; (iii) drying the extrudates in flowing N2 at 900° F. (482° C.); and (iv) calcining the dried extrudates at 1000° F. (538° C.) in air. The thus produced transalkylation catalyst compositions were then measured for surface areas and pore volumes, which are reported in Table 1 below. The comparative catalyst composition in comparative Example 1 comprises the base zeolite. As can be seen from Table 1, the transalkylation catalyst compositions in Examples 2, 3, and 4 exhibited increasingly higher mesopore surface areas than that of Example 1. U.S. Patent Application Publication No. 2013/0183231 teaches methods for varying mesoporous surface areas of zeolites.

TABLE 1

| | | Micropore | Mesopore | BET | Total |
|---|---|---|---|---|---|
| | Ammonia | Surface | Surface | Surface | Pore |
| Example | Adsorption | Area | Area | Area | Volume |
| # | (meq/g) | (m²/g) | (m²/g) | (m²/g) | (cm³/g) |
| 1 | 0.51 | 549 | 180 | 729 | 0.56 |
| 2 | 0.51 | 374 | 337 | 711 | 0.61 |
| 3 | 0.50 | 370 | 426 | 796 | 0.65 |
| 4 | 0.48 | 282 | 486 | 768 | 0.66 |

### Examples 5 to 9

In Examples 5-8, a commercially available low activity faujasite zeolite was used as a base zeolite (a precursor catalyst composition), which was treated to produce three different batches of treated faujasite crystals with varying mesopore surface areas. Four different transalkylation catalyst compositions were produced by (i) mixing each of the base zeolite and the three treated batches of zeolite with an identical commercially available alumina binder, with a zeolite/binder weight ratio of 65/35; (ii) extruding the mixture into 1/20″ quadralobes; (iii) drying the extrudates in flowing N2 at 900° F. (482° C.); and (iv) calcining the dried extrudates at 1000° F. (538° C.) in air. In Example 9, a commercially available beta zeolite was used as a base zeolite and treated to alter surface areas thereof. In a manner identical to Examples 6-8 described above, a catalyst composition for Example 9 was made from the treated zeolite and the alumina binder. The thus produced transalkylation catalyst compositions in Examples 5-9 were then measured for surface areas and pore volumes, which are reported in Table 2 below. The comparative catalyst composition in comparative Example 5 comprises the low activity faujasite base zeolite. As can be seen from Table 2, the transalkylation catalyst compositions in Examples 6, 7, and 8, comprising the treated zeolites, exhibited increasingly higher mesopore surface areas than the comparative catalyst composition of comparative Example 5.

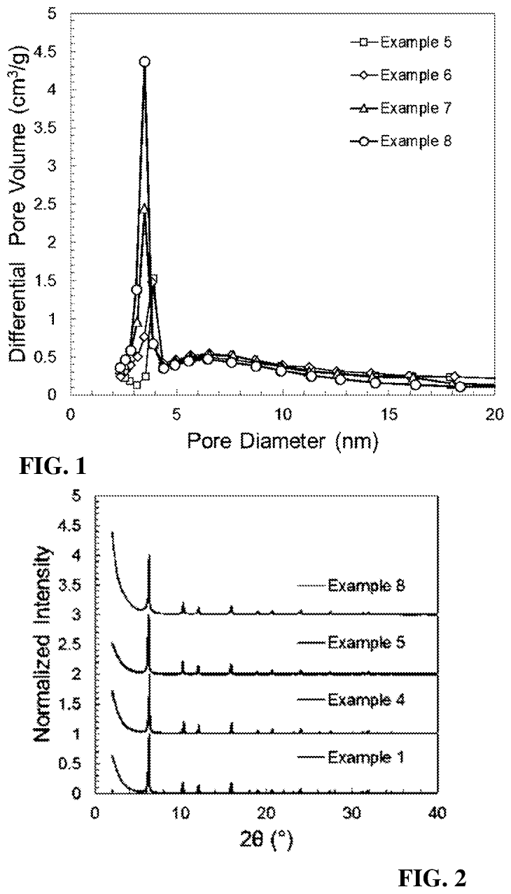

The properties of the comparative catalyst composition of Example 5 and the inventive transalkylation catalyst compositions of Examples 6 to 8 are summarized in Table 2. In addition, FIG. 1 displays a graph of the differential pore volume against the pore diameter for the catalyst compositions of Examples 5 to 8. As can be seen, all four catalyst compositions display a similar sharp pore at ˜3.5 nm which increases with increasing mesoporosity. X-ray diffraction (XRD) patterns of the comparative catalyst composition of comparative Example 1, the transalkylation catalyst composition of inventive Example 4, the comparative catalyst composition of comparative Example 5, and the transalkylation catalyst composition of inventive Example 8 are shown in FIG. 2, from which it can be seen that the transalkylation catalyst compositions of Examples 4 and 8 exhibit similar long range order to those of comparative Examples 1 and 5, respectively, as evidenced by similar XRD peaks.

TABLE 2

| | | Micropore | Mesopore | BET | Total |
|---|---|---|---|---|---|
| | Ammonia | Surface | Surface | Surface | Pore |
| Example | Adsorption | Area | Area | Area | Volume |
| # | (meq/g) | (m²/g) | (m²/g) | (m²/g) | (cm³/g) |
| 5 | 0.31 | 690 | 142 | 832 | 0.452 |
| 6 | 0.27 | 531 | 292 | 823 | 0.468 |
| 7 | 0.28 | 425 | 406 | 831 | 0.542 |
| 8 | 0.29 | 325 | 495 | 820 | 0.578 |
| 9 | 0.34 | 271 | 341 | 612 | 0.875 |

### Example 10

One gram of each of the transalkylation catalyst compositions of Examples 1 to 9 was sized with a No. 14/25 mesh using a mortar and pestle and then mixed with 1 g of quartz before being loaded into a 16-channel packed bed flow reactor that can modulate the temperature of its 4 modules (4 channels/module) independently. The reactor was then used to conduct transalkylation tests on a feed mixture of benzene, DIPB and TIPB having the composition shown in Table 3. The feed components were acquired from Aldrich, mixed, and then filtered with three separate molecular sieve materials. The overall feed composition was determined by GC.

TABLE 3

| Component | Mass Fraction |
|---|---|
| Benzene | 0.61 |
| 1,3-Di-isopropylbenzene | 0.16 |
| 1,4-Di-isopropylbenzene | 0.13 |
| 1,3,5-Tri-isopropylbenzene | 0.10 |

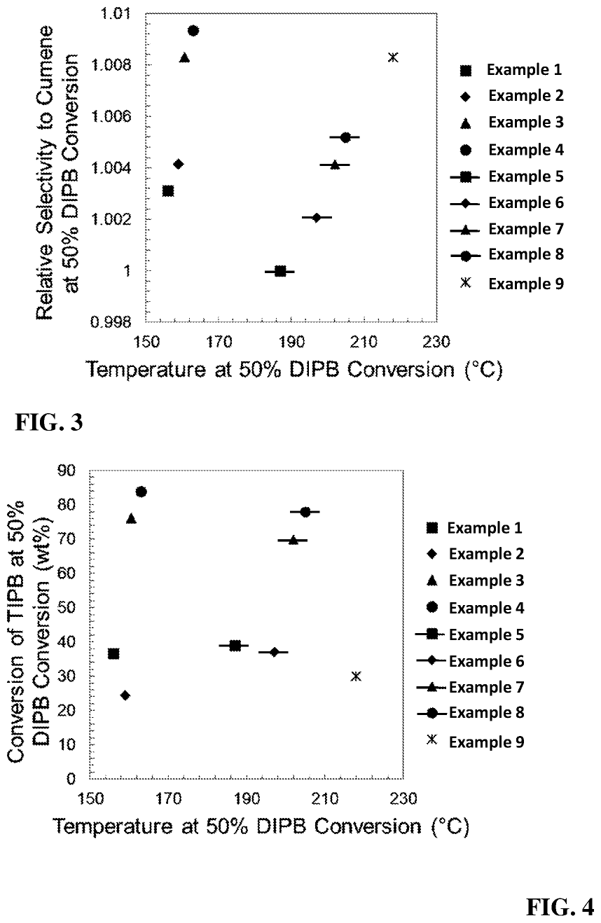

In the transalkylation tests, the temperature of each module was modulated in order to achieve 50% conversion of DIIPB and this temperature was then used as a benchmark for activity. The reactants and products were liquid phase and each reactor was held at a pressure of 300 psig (2170 kPa) with a flow rate of 1.82 cc/min, which corresponds to a weight hourly space velocity (WHSV) of 6. The results of the tests are summarized in FIGS. 3 and 4.

FIG. 3 shows the relative selectivity for cumene of the different transalkylation catalyst compositions at the temperature at which 50% conversion of DIIPB occurs. As can be seen, the comparative catalyst compositions (Examples 1 and 5) display lower selectivity to cumene (with ethylbenzene and n-propylbenzene as common side products) compared to the samples with greater mesoporosity. Generally, within a given subset of materials, increased mesoporosity yields a more selective catalyst.

FIG. 4 displays the relative conversion of TIPB at the temperature at which 50% DIPB conversion was obtained (DIPB conversion usually dictates the normal operating condition). As can be seen, the transalkylation catalyst compositions of Examples 6-9 showed step-out behavior compared to their non-mesoporous analogue. As mesoporous surface areas of the catalyst compositions gradually increased from Example 5 to 6, to 7, and to 8, conversion of TIPB to cumene increased. The catalyst compositions with increased mesoporous surface areas offer the opportunity to operate the transalkylation bed at much lower temperatures or at increased WHSV rates without necessitating purge cycles to get rid of unreacted TIPB molecules.

While this disclosure has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of this disclosure.

1. A transalkylation process, the process comprising:
(I) providing a precursor catalyst composition exhibiting a first mesoporous surface area of a 1 m²/g,;
(II) treating the precursor catalyst composition to obtain a treated precursor catalyst composition, wherein the treated precursor catalyst composition exhibits a second mesoporous surface area of a2 m²/g, and wherein a2>a1;
(III) Tip forming a transalkylation catalyst composition from the treated precursor catalyst composition;
(IV) feeding a transalkylation feed mixture comprising (i) benzene and (ii) di-alkylated benzene(s) and/or tri-alkylated benzene(s) into a transalkylation zone; and
(V) contacting the transalkylation feed mixture with the transalkylation catalyst composition in the transalkylation zone under transalkylation conditions to produce a transalkylation effluent rich in mono-alkylated benzene(s) relative to the transalkylation feed mixture.

2. The transalkylation process of claim 1, wherein the precursor catalyst composition comprises a zeolite.

3. The transalkylation process of claim 2, wherein the zeolite is selected from the group consisting of FAU, BEA, MOR, and MWW framework zeolites, and mixtures and combinations thereof.

4. The transalkylation process of claim 1, wherein the zeolite is a FAU framework zeolite.

5. The transalkylation process of claim 1, wherein 10%≤(a2−a1)/a1*100%≤1000%.

6. The process of claim 1, wherein step (II) comprises:
(IIa) treating the precursor catalyst composition with a surfactant.

7. The process of claim 6, wherein the surfactant is selected from the group consisting of cetyltrimethylammomium bromide, cetyltrimethylammonium chloride, and mixtures thereof.

8. The process of claim 6, wherein step (II) further comprises:
(IIb) treating the precursor catalyst composition with an acid before or after step (IIa).

9. The process of claim 8, wherein the acid is selected from the group consisting of hydrochloric acid, sulfuric acid, nitric acid, acetic acid, sulfonic acid, oxalic acid, citric acid, ethylenediaminetetraacetic acid, tartaric acid, malic acid, glutaric acid, succinic acid, and mixtures thereof.

10. The process of claim 6, wherein step (II) further comprises:
(IIc) treating the precursor catalyst composition with a base after step (IIa) and/or step (IIb).

11. The process of claim 10, wherein the base is selected from the group consisting of NaOH, NH₄OH, KOH, Na₂CO₃, TMAOH and mixtures thereof.

12. The process of claim 1, wherein the transalkylation conditions include a temperature in the range from about 100° C. to about 300° C.

13. The process of claim 1, wherein the transalkylation conditions include a temperature in the range of about 150° C. to about 220° C.

14. The process of claim 1, wherein the mono-alkylated benzene comprises cumene.

15. The process of claim 1, wherein the transalkylation catalyst composition further comprises a binder.

16. A process for producing a mono-alkylated benzene, the process comprising:
(a) contacting a feedstream comprising benzene with an alkylating agent in the presence of an alkylation catalyst composition under alkylation conditions effective to convert at least part of the benzene in the feedstream to the desired mono-alkylated benzene and produce an alkylation effluent comprising the mono-alkylated benzene, di-alkylated benzene(s) and tri-alkylated benzene(s);
(b) separating the alkylation effluent into a first fraction containing the mono-alkylated benzene and a second fraction containing di-alkylated benzene(s) and tri-alkylated benzene(s);
© contacting at least part of the second fraction with benzene in the presence of a transalkylation catalyst composition under transalkylation conditions including a temperature from 100 to 300° C. effective to convert at least part of the di-alkylated benzene(s)and tri-alkylated benzene(s) to the mono-alkylated benzene and produce a transalkylation effluent, wherein the transalkylation catalyst composition is obtained by:
(c1) providing a precursor catalyst composition exhibiting a first mesoporous surface area of a1 m²/g;
(c2) treating the precursor catalyst composition to obtain a treated precursor catalyst composition, wherein the treated precursor catalyst composition exhibits a second mesoporous surface area of a2 m²/g, and wherein a2>a1; and
(c3) forming a transalkylation catalyst composition from the treated precursor catalyst composition; and
(d) recovering the mono-alkylated benzene front the transalkylation effluent.

17. The process of claim 16, wherein x1%≤(a2−a1)/a1*100%≤x2%, where xand x2, can be, independently, e.g., 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600 700, 800 900, 1000, as long as x1<x2.

18. The process of claim 16, wherein the alkylation catalyst composition comprises one or more zeolites selected from the group consisting of BEA and MWW structure types.

19. The process of claim 16, wherein the alkylating agent comprises propylene and/or isopropanol and the mono-alkylated benzene comprises cumene.

20. The process of claim 16, wherein the transalkylation catalyst composition further comprises a binder.

21. The process of claim 16, wherein the alkylation effluent further comprises unreacted benzene and the process further comprises:
© separating the unreacted benzene from the alkylation effluent and recycling at least part of the unreacted benzene to the contacting (a) and/or the contacting (c).

22. The process of claim 16, wherein the transalkylation effluent further comprises unreacted benzene and the process further comprises:
(f) separating the unreacted benzene from the transalkylation effluent and recycling at least part of the unreacted benzene to the contacting (a) and/or the contacting (c).

23. The process of claim 16, wherein the feedstream further comprises impurities and the process further comprises:
(g) contacting the feedstream with an absorbent under conditions effective to remove at least part of the impurities, wherein the impurities comprise compounds having at least one of the following elements: nitrogen, halogens, oxygen, sulfur, arsenic, selenium, tellurium. phosphorus, and Group 1 through Group 12 metals.
